(19) United States
(12) Patent Application Publication
(10) Pub. No.: US 2005/0171492 A1
(43) Pub. Date: Aug. 4, 2005

(54) Coding device for identifying medical lines

(76) Inventor: Joel J. Rodriquez, Gloucester, MA (US)

Correspondence Address: FOLEY & LARDNER LLP, 111 HUNTINGTON AVENUE, 26TH FLOOR, BOSTON, MA, 02199-7610, US

(21) Appl. No.: 11/013,281

(22) Filed: Dec. 15, 2004

Related U.S. Application Data
(60) Provisional application No. 60/529,795, filed on Dec. 16, 2003.
(60) Provisional application No. 60/542,747, filed on Feb. 6, 2004.
(60) Provisional application No. 60/545,880, filed on Feb. 19, 2004.

(51) Int. Cl. A61M 25/00

(52) U.S. Cl. 604/264

(57) ABSTRACT

The present invention provides devices for identifying medical lines. The devices provide for rapid visual identification of the medical line, and are particularly useful in field situations or emergency situations. Both illuminated and non-illuminated devices are described, the former particularly useful in low ambient light environments.

### RELATED APPLICATIONS

The present application is related to and claims the benefit of provisional Application Ser. No. 60/529,795, filed Dec. 16, 2003, entitled “Intravenous Line and Tube Coding Device and Kit”, provisional Application Ser. No. 60/542,747, filed Feb. 6, 2004, entitled “Disposable Point of Infusion Lighting Device” and provisional Application Ser. No. 60/545,880, filed Feb. 19, 2004, entitled “Visual Enhancing System and Kit for Intravenous Line and Tubes”. Each application is hereby incorporated herein by reference in its entirety.

### FIELD OF THE INVENTION

The present invention relates to the field of medical equipment, and the illumination and control of the same.

### BACKGROUND OF THE INVENTION

The administration of medication, solutions and nutrients to patients via intravenous administration is a well established medical practice. In medical settings, patients are often treated by introducing prescribed medication or other prescribed liquids directly into the blood stream of the patient. This can be accomplished by direct instillation through an intravenous administration device.

A typical intravenous administration system consists of a length of sterile flexible plastic intravenous tubing. One end of the tubing is adapted to a supply reservoir such as a syringe or bag, typically containing a prescribed medication or other prescribed liquid. The other end of the tubing is adapted to be inserted into a venous blood vessel of the patient via a needle, or is capable of insertion into a device known as an infusion port, that is in turn coupled to a needle inserted appropriately into the patient. An infusion port is typically left in the patient for a prolonged period, and provides for a route of intravenous administration of one or more types of medications over a period of time, through a common injection site, for example intravenous (IV) drips provide the patient with continuous administration of saline and nutritional substances, and medications and other substances can be administered intermittently through the same port or through the same IV drip.

It is common in medicine, especially in an emergency room setting, for patients to receive simultaneous, multiple intravenous medications or solutions through intravenous tubing. The simultaneous use of multiple intravenous fluid lines has led to some major problems in the medical field.

The number of IV feeds and catheter lines in an operating theater, an intensive care room or emergency room can result in confusion to a medical practitioner as to which IV connection routes to particular insertion sites. Multiple intravenous tubes are long and transparent and frequently become entangled, intertwined or twisted, making it even more difficult for the medical care provider to determine which medication is flowing through which intravenous tube. Safety, timely delivery of medication, and efficiency are paramount to critical care. In routine emergency situations or during surgical procedures, quick identification of a medicinal fluid source is often required, but even in more simplistic and less time intensive procedures, accuracy remains critical. For example, administration of incorrect medication into a epidural line instead of an intravenous one would likely result in the death of the patient.

A number of devices and efforts were made to provide health care worker with systems for more rapidly identifying a medicament flow through an intravenous line. The references are briefly described, and the entirety of each disclosure is intended to be incorporated herein by reference. U.S. Pat. No. 6,059,768 to Friedman discloses a coding system for intravenous tubing, where the tubing has a secondary axial cavity extending continuously along substantially an entire length of the tubing wall for visually distinguishing the tubing, the cavity being filled with a chemiluminescent material. This type of tubing is expensive to manufacture and use, and if the integrity of the tubing is compromised, the chemiluminescent solution may contaminate the medication. U.S. Pat. No. 6,613,012 to Kraushaar discloses a matched set of plural identification elements bearing matching identification symbols, wherein a first one of the set is attachable to the IV container; a second one of the set is attached to the drip chamber; and a third one of the set is attached to the IV line remote from the first and second identification elements in the set. U.S. Pat. No. 5,974,708 to Webb et al. discloses an intravenous line identification system that includes a plurality of sets of at least two identifying tags, each set used for identifying a medicinal source and a medicinal output for one of a plurality of intravenous lines. Each set is provided with a highly visible color distinct from every other set. Each tag in a set is coupled to another tag in the same set by at least one frangible or decouplable connection. Each tag further has an opening enabling the tag to be inserted over the intravenous line, and a preferably circular hole, about which the tag holds the intravenous line. For each intravenous line, the tags are applied over the intravenous line as a set, i.e., while coupled. Once on the intravenous line the tags are decoupled from each other and slid in opposite directions along the line toward the medicinal source and output.

However, there remains a need for an improved intravenous identifying system that can provide enhanced visual identification of medical lines.

### SUMMARY OF THE INVENTION

It is an object of the present invention to provide an identification system that enables enhanced visual identification of the plurality of lines used during medical procedures. It is another object of the invention to provide an identification system that can be easily and quickly applied to the various lines. It is yet another object of the invention to provide a line identification system, that provides enhanced visual identification of the lines in low ambient light situations.

The present invention achieves these objectives as it provides a coding system that enables a user to have instant visual identification, confirmation or clarification as to the identity of one or more medical lines or tubes, e.g., i.v. lines, catheters and the like, that are conduits between a patient and the patient's respective medication supply (or other fluid), or to medical equipment and devices. In a preferred aspect, the coding system is applied to medication lines. In one embodiment, the medication lines are intravenous lines, which provide for fluid communication from medication sources to the veins of the patient, and the code system thus provides for identification of specific lines used to deliver each specific medication. In another embodiment, the medication line is a epidural line, which provides for fluid communication from medication sources to the intraspinal spaces of the patient, and the code system thus provides for identification of this line, and further provides a signal that only specific medications are to be introduced through this particular line.

In another aspect, the coding system is applied to catheter lines. In one embodiment, the catheter is an angioplasty catheter, and the coding device provides for identification of the lines that are adapted to the inflation/deflation source. In another embodiment, the catheter is a bladder catheter, and the coding system provides for identification of the line that is adapted to a medical waste container.

In yet another aspect, the coding system is applied to medical devices lines. In various embodiments, these include, for example but not limited to, irrigation lines, aspiration and suction lines, gas supply lines, and electrical lines such as those attaching to monitors.

The medical line coding device of the present invention includes a housing and an illumination source; the housing having a first channel for the illumination source, and further comprising a second channel through which a medical line can pass. In one embodiment, the housing is capable of folding over itself. In another embodiment, the housing further includes geometric features that engage and hold each other when the housing is folded over itself. In still another embodiment, the engaged geometric features irreversibly lock the housing in a closed position. In even another embodiment, the illumination source is chemiluminescent, and provides light of a particular color. In yet another embodiment, the illumination source provides light of two or more particular colors.

In another aspect, the invention provides, a method of identifying a medical line by: obtaining a medical line coding device comprising a housing and an illumination source; the housing having a first channel for the illumination source, and further comprising a channel through which a medical line can pass; isolating a medical line to be identified; and activating the illumination source and affixing the medical line coding device to a medical line, thereby identifying the medical line.

In yet another aspect, the invention provides a kit for identifying a medical line the kit including, a medical line coding device further comprising, a housing and an illumination source; the housing having a first channel for the illumination source, and further comprising a second channel through which a medical line can pass; the kit further comprising suitable packaging material and instructions for using the device to identify a medical line. The kit contents are preferably sterile. In one embodiment, the packaging material provides a hermetic seal that indicates the sterility of the device contained therein.

In even another aspect, the invention provides a method of making a kit for identifying a medical line comprising, obtaining a medical line coding device further comprising, a housing and an illumination source; the housing having a first channel for the illumination source, and further comprising a second channel through which a medical line can pass; obtaining suitable packaging material and instructions for using the device to identify a medical line, and incorporating the device into the packaging material.

In even yet another aspect, the invention provides a method of making a kit for identifying a medical line comprising, obtaining a system for identifying a medical line comprising one or more medical line coding devices, each device comprising, a housing and an illumination source; the housing having a first channel for the illumination source, and further comprising a second channel through which a medical line can pass, wherein the illumination source provides a color, which is interpretable as coding for a particular medication or fluid to be administered through the identified line.

These and other features, aspects, and advantages of the present invention will become better understood with regard to the following description, appended claims, and the accompanying drawings.

### SUMMARY OF THE DRAWINGS

FIG. 1 is an illustration of a non-illuminated medical line identification and coding device;

FIG. 2 is an illustration of an illuminated medical line identification and coding device;

FIG. 3 is a three dimensional view of the device shown in FIG. 2;

FIG. 4 is an end view of the illuminated medical line identification and coding device;

FIG. 5 is a side view of the illuminated medical line identification and coding device.

### DETAILED DESCRIPTION

The present invention is directed to a coding system, that provides for enhanced visual identification of a single critical line, or a plurality of lines and tubes used during medical procedures. The coding system can be easily and quickly applied to the lines. An adaptation of the invention permits an illumination source to be integrated into the system, the illumination source providing enhanced visual identification of the medication lines in low ambient light situations. The present invention provides a coding system that enables a user to have instant visual identification, confirmation or clarification as to the identity of one or more medical lines or tubes, e.g., i.v. lines, catheters and the like, that are conduits between a patient and the patient's respective medication supply (or other fluid), or to medical equipment and devices.

As used herein, a medical line refers to a conduit having two terminal ends, one that is adapted to a medical device, and the second end capable of direct insertion into a patient or capable of adaptation to a second medical device that is directly inserted into the patient. A nonlimiting example of a medical line that is adapted to a first and a second medical device is a medication line, such as an i.v. drip, where the line provides for fluid communication between a medication source and a needle inserted into the patient. A nonlimiting example of such a medical line that is adapted to a medical device and is directly inserted into a patient is a catheter, which is typically a hollow flexible tube for insertion into a body cavity, duct, or vessel to allow the passage of fluids or distend a passageway.

In a preferred aspect, the coding system is applied to medical lines used for the administration of medications, nutrients, or fluids to the patient. Such lines can be intravenous lines, which provide for fluid communication from medication sources to the veins of the patient, wherein the code system provides for identification of specific lines used to deliver each specific medication. Other such lines include an epidural line, which provides for fluid communication from medication sources to the intraspinal spaces of the patient, wherein the code system provides for identification of this epidural line, and further provides a signal that only specific medications are to be introduced through this particular line. The invention is also useful with medical lines attached to devices, such as an angioplasty catheter, wherein the coding device provides for identification of the lines that are adapted to the inflation/deflation source. Alternatively, where the catheter is a bladder catheter, the coding system provides for identification of the line that is adapted to a medical waste container. Similarly, the coding system is applied to medical lines adapted to medical devices for example but not limited to, irrigation lines, aspiration and suction lines, gas supply lines, and electrical lines such as those attaching to monitors. A skilled artisan will recognize that the function of the medical line does not limit the applications for the invention, which can be used with virtually any tube, conduit, line, hose, or wire.

The coding system of the present invention includes illuminated and non-illuminated embodiments.

A non-illuminated medical line coding system includes one or more cylindrical transparent or semitransparent lenses that can be removably attached to a medical line. Preferably, the lenses are constructed from plastic, or a similar material that can be formed, molded or extruded. A preferred material will be flexible enough to conform to shape of the medical line, but with some structural rigidity such that the device substantially returns to its original shape after deformation. The device thus operates as a sleeve, which wraps around the medical line. The device can be various sizes. The interior diameter of the device will preferably approximate the exterior diameter of the line for which it will be used, and while some variation in tolerances in acceptable, if the device is too large, it will not secure itself to the line, and if too small, it may impede the flow of liquids through the line. The length of the device is less important, but there is a relationship between the length of the device and its usefulness as an identification source. By way of illustration, a long device, e.g., six inches in length or more, will provide a very visible indicator, and a short device e.g., less than one inch in length, provides less visible impact. It is preferred that the device generally be about 1-2 inches in length, more or less, which provides acceptable visual impact without significant physical coverage of the medical line. Preferably the lenses are colored or tinted, and may further include writing or symbols.

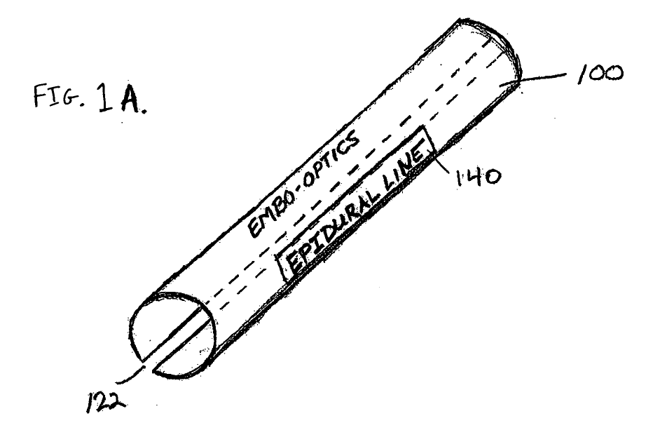

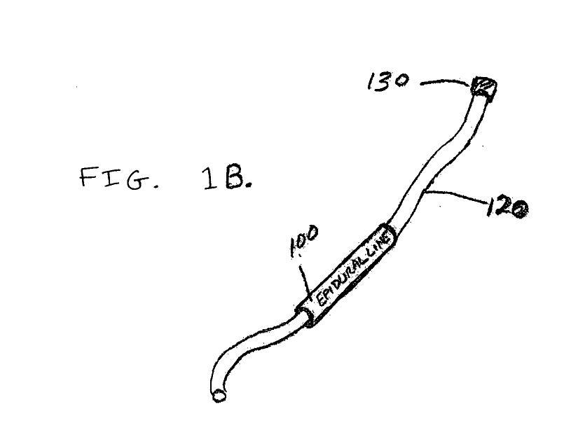

Referring now to FIG. 1, a non-illuminated medical line coding device is shown as FIG. 1a, which is illustrated in use on a typical medical line shown in FIG. 1b as an epidural line. The medical line coding system includes a cylindrical transparent-colored lens 100, that can be removably attached to one or more locations on the intravenous line 120. Preferably the device is positioned close to a port 130, such that it is immediately visible to a medical provider who is administering medication through the port. The lens 100 includes a slit 122 along its axial length. A user of the device inserts the medical line 120 into the slit 122, whereby the line is introduced into the center of the cylinder, and the lens thus wraps around the exterior of the medical line. Friction between the exterior surface of the line and the interior surface of the lens secures the device to the medical line. The slit 112 allows for expansion of the device by a user, thereby decreasing the frictional force, permitting the user to slide the device along the length of the medical line thereby repositioning the device. The coding system can include sets of same-color individual lens devices that can be placed at multiple locations along the length of the line. Alternatively, matched sets of multiple color lenses can be provided, for use with multiple medical lines. In a preferred aspect, the devices are used in matched pairs. In addition to color, the device may have imprinted data or formed features such as nomenclatures, icons, symbols, warnings, etc. Icons indicating function are preferred for use in critical care and first response situations. In the present illustration, the device includes a region 140 bearing the legend “Epidural Line”, warning the user that the medical line is an epidural line. In this embodiment, the lettering is raised, and is permanently formed into the device during the molding process.

In an alternative aspect, the invention provides an illuminated medical line coding system. The illuminated medical line coding system is preferably a self-illuminating device that provides visual identification of medical lines in low ambient light environments, common in many surgeries, hospital wards, and field environments. The illuminated medical line coding system is affixed along the length of a medical line, thereby identifying the line and distinguishing it from other lines. The self-illuminating device also provides a source of localized illumination, wherein medical devices and controls in proximity to the device, i.e., within the luminescent field, are thereby illuminated. If the device presents imprinted data or formed features (as described above), such are preferably illuminated by the illumination source.

An illuminated medical line coding system includes a housing and an illumination source. The housing provides a structural frame that is adapted to receive and contain an illumination source. Preferred materials for the housing include polypropylene, polyethylene, and other common plastics. The housing can be transparent or semitransparent, or can be opaque provided it has one or more apertures or windows, through which a user may view the illumination source, or light therefrom. Alternative embodiments having variously sized and shaped apertures are suitable for applications where it is desirable to direct light emissions from the housing to a particular illumination field. The housing has one or more geometric features that provide a reversible clamp or irreversible locking mechanisms, and the housing unit is designed to fold upon itself, thereby bringing the geometric features into proximity whereby they engage each other and lock the housing in a closed position. In such a closed position, the illumination source is secured inside the housing. The housing has a semicircular groove or channel, or alternatively a series of apertures in the structural frame, such that the housing can receive the medical line. The appropriate diameter of the channel will depend on the outside diameter of the medical line as described above. The housing, when folded upon itself, will encompass and contain the medical line, in such manner that the line passes through the illuminated medical device. In a preferred embodiment, the device is irreversibly attached to the medical line. In various other embodiments, the housing includes words or symbols or other identifying and distinguishing features, which can be inscribed upon or formed into the housing.

The illuminated medical line coding system includes an illumination source. The illumination source can be, for example, an LED or bulb. Controls and power supplies would thus be provided, which can be remote, or integrated into the housing. Alternatively, an optical fiber can be introduced into the device. But preferably, the illumination source is a self contained unit. A small chemiluminescent device is a suitable self-contained illumination source, and provides a bright cool light for many hours. A Cyalume®-type chemical light stick exemplifies such a chemiluminescent device. The user activates the illumination source, e.g. bends the stick, prior to insertion of the illumination source into the housing. The illumination source preferably emits a colored light, thus providing illumination but also permitting rapid visual identification of the line, e.g., red for an epidural catheter line, green and blue for a set of intravenous medication lines, and yellow for a medical waste catheter. Chemiluminescent devices that emit various color spectra are commonly available, such as those from OmniGlow, Inc. In certain embodiments, a single illumination source is employed, that provides two or more discrete regions of color, such as dual color chemical light sticks. Color lenses may also be incorporated into one or more regions of the housing to provide additional multi-color effects.

The present invention further provides for coding devices that function as point of infusion illumination sources. An illuminated device is attached to a medical line proximal to the point of infusion, i.e., near the needle, port, or close to the body of the patient. Preferably, the illumination source includes a diffusing lens, to provide flood lighting of the infusion area. Advantageously, direct lighting of the infusion point (or region) aids medical personnel in locating the veins of a patient, which can be difficult to locate in small children, persons with darker skin pigmentation (e.g., African Americans, Hispanics, Indians) and persons having extremely light skin (persons of Northern European descent). The point of infusion lighting source allows light to travel through the skin of the patient, thereby illuminating or highlighting the veins. Color spectrum of the illumination source can be selected to more particularly distinguish blood vessels, e.g., blue or white instead of red. For other procedures, such as catheterization in low ambient light environments, the device provides a localized light source. Pair sets of illuminated medical devices, attached to the medical line near the point of infusion and proximal to the medication source, are preferred. If the device is intended to be removable from the medical line, the housing is modified accordingly, e.g., spring clips are incorporated into the housing instead of locking tabs.

The illumination and identification systems described are suitable for incorporation into a kit for surgical and medical uses. A coding system kit includes any of the following: one or more illuminated medical line coding devices, one or more non-illuminated medical line coding devices, or both illuminated and non-illuminated devices. The kit further includes suitable packaging materials and instructions for use of the illuminated and non-illuminated medical line coding devices. Kit contents may be sterilized, in which case the packaging material is preferably hermetically sealed. Sterilization techniques for medical devices are well known in the art, for example, irradiation is suitable for plastic coding devices. Kits preferably include multiple color sets (illumination sources or lenses). Pair sets of colors are particularly preferred.

### EXAMPLE ONE

### An Illuminated Medical Line Coding Device

An illuminated medical line coding device is provided. The device is intended to use a chemiluminescent light stick as the illumination source, and is designed to irreversibly lock on to a medical line. The device is referred to as a Flush Light™, (alternatively as a Flush Lite™). The device includes a housing and an illumination source. The housing has a first channel for the illumination source, and a second channel through which the medical line can pass.

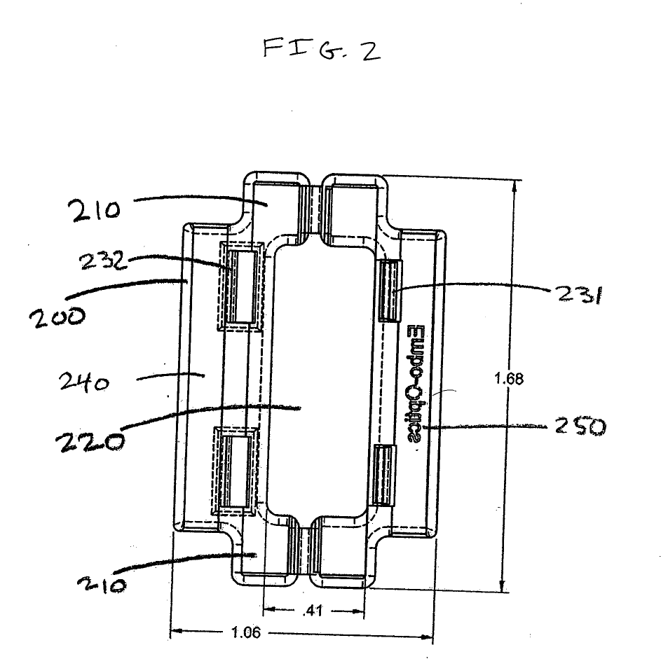

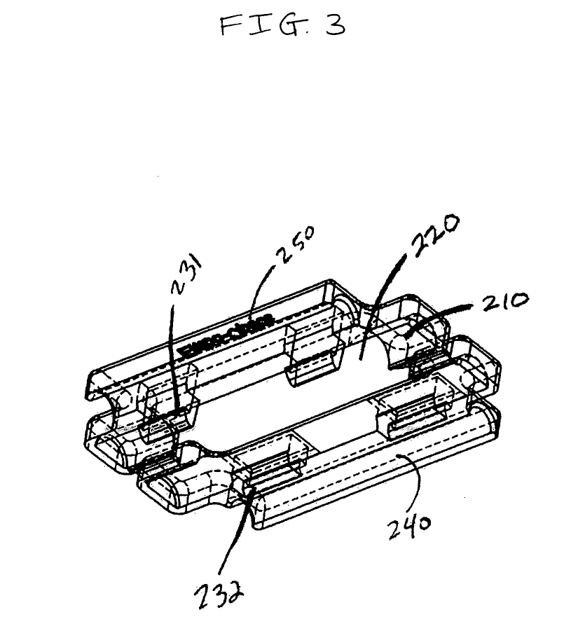

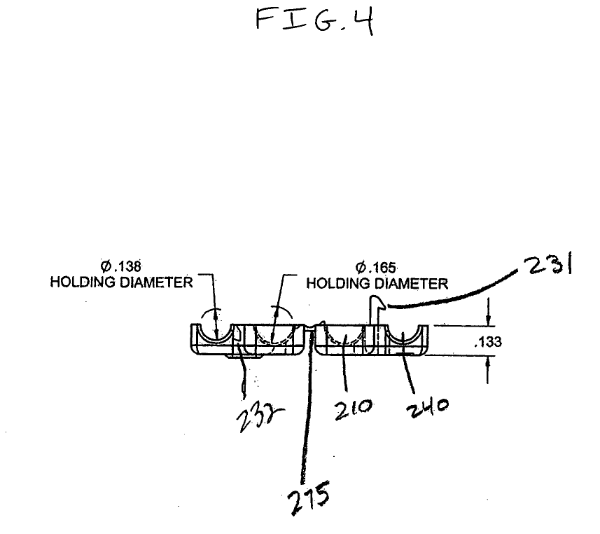

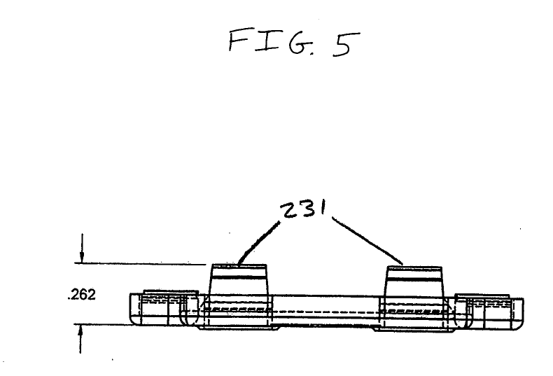

Referring now to FIG. 2, the illuminated medical line coding system is shown in the open position. The device includes a plastic housing 200 having a first channel 210, that is capable of receiving and containing the illumination source (not shown). The housing 200 may include at one or more points adjacent to the first channel, geometric features that provide a convenient snap fit for the illumination source. The housing contains a central aperture 220 that permits light from the illumination source to escape from the housing. The housing 200 has a second channel 240 for the medical line. The housing additionally includes one or more paired features (231 and 232) that engage when closed, and thereby permit the device to lock in the closed position when applied to a medical line. The housing has at least one region 250 that provides for identifying information. As shown in FIG. 2, the information provided thereon is the manufacturers trademark Embo-Optics™ but as described above, other identifying information can be used, e.g., type of medical line. FIG. 3 illustrates a three-dimensional view of the device, again in an opened position. FIG. 4 illustrates an end view of the device. The paired features 231 232 are visible as a tab 231 that engages an extension 232 when the tab is brought into proximity with the edge. The first channel 210 for the illumination source is shown, and in this embodiment adapted to receive a cylindrical chemiluminescent light stick having an outside diameter of approximately 0.165 inches or less. A second channel is also shown, adapted to receive a medical line having an outside diameter of approximately 0.138 inches or less. The dimensions provided are for nonlimiting illustration, and are variable depending e.g., on the diameter of the medical line and the illumination source. The device as shown in FIG. 4 includes a hinge 275, allowing the housing to fold over. FIG. 5 shows a side view of the device. The locking tabs 231 are visible.

To use the Flush Light™ illuminated medical line coding device, a user first activates a chemical light stick (provided with the housing) by bending it, thereby breaking the internal compartment, and briefly shakes it, thereby mixing the solutions. The activated illumination source is placed into the first channel of the housing. The device is then applied to a position on a medical line. The user places the device on the medical line preferably near a port or supply, orienting the device so that the medical line lies inside the second channel. The user folds the housing over the medical line, engaging the locking tabs. Frictional forces between the interior surfaces of the housing channel and the exterior of the medical line hold the device on the line. The medical line is thereby identified with a colored light source, which is clearly visible through the aperture of the device. Optionally, a user can write on the Flush Light™ illuminated medical line coding device, or can apply a self-adhesive label to the device, providing additional identifying information such as the chemical make-up of the solutions to be administered through the identified medical line, or warning information, or the like.

### EXAMPLE TWO

### A System for Coding Medical Lines

A kit for use in a medical procedure is described herein, providing a user with a system for the rapid visual identification of medical lines. The system is referred to as an IV ID Code Kit™ and is suitable for distribution throughout a medical facility, for use with patients undergoing medical procedures that involve a critical medical line or a plurality of medical lines. While the system described is referred to as an IV ID Code Kit™, it should be pointed out that this system is intended to identify non-intravenous lines (e.g., catheters and electrical lines) as well as i.v. medical lines.

The IV ID Code Kit™ minimally provides in one embodiment, an illuminated medical line coding device, suitable instructions for using the identification system and the individual devices therein, and, suitable packaging (for example a blister pack). Preferably the kit includes a pair set of illuminated medical devices. Pair sets are color matched. Housings are plastic, which can be color matched to the color spectrum of the illumination source. The housing of the illuminated devices have a substantially flat area designated for marking, e.g., with an indelible marker, and thereby further identify specific features of the solutions and medications being dispensed through the particular line. By way of illustration, the device pairs for use with an epidural catheter have formed warning features and are illuminated, for example, red. The device pairs for use with a bladder catheter has imprinted thereof, a medical waste indication and are illuminated, for example, yellow. The device pairs for use with an i.v. drip are marked before use with the solution contents and are illuminated, for example, blue. The device pairs for use with secondary i.v. lines are illuminated, for example, green.

The kit can be supplied as a sterile, hermetically sealed package, having an apparent seal that must be affirmatively broken by the user, thereby indicating the contents have been exposed to the environment. Included with the kit are such optional other items as, non-illuminated medical line coding devices, labels for enhanced identification of a medical line or the substance therein, an indelible marker and spare or redundant components, e.g., extra illumination sources to provide localized light fields for the user.

Equivalents

From the foregoing detailed description of the specific embodiments of the invention, it should be apparent that unique medical devices and identification systems have been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims. For instance, the choice of color spectrum, or the illumination source used is believed to be matter of routine for a person of ordinary skill in the art with knowledge of the embodiments described herein.

1. A medical line coding device comprising, a housing and an illumination source; the housing having a first channel for the illumination source, and further comprising a second channel through which a medical line can pass.

2. The device of claim 1, wherein the housing is capable of folding over itself.

3. The device of claim 2, wherein the housing further comprises geometric features that engage and hold each other when the housing is folded over itself.

4. The device of claim 3, wherein the engaged geometric features irreversibly lock the housing in a closed position.

5. The device of claim 1, wherein the illumination source is chemiluminescent.

6. The device of claim 1, wherein the illumination source provides light of a particular color.

7. The device of claim 1, wherein the illumination source provides light of two or more particular colors.

8. A method of identifying a medical line comprising:
a. obtaining a medical line coding device comprising a housing and an illumination source; the housing having a first channel for the illumination source, and further comprising a channel through which a medical line can pass;
b. isolating a medical line to be identified; and
c. activating the illumination source and affixing the medical line coding device to a medical line, thereby identifying the medical line.

9. The method of claim 8, wherein the illumination source provides a color, which is interpretable by the user, as coding for a particular medication or fluid that is appropriately administered through the identified line.

10. The method of claim 8, wherein the illumination source is chemiluminescent.

11. The method of claim 8, wherein the illumination source provides light of a particular color.

12. The method of claim 8, wherein the illumination source provides light of two or more particular colors.

13. The method of claim 8, wherein the housing is capable of folding over itself.

14. The method of claim 13, wherein the housing further comprises geometric features that engage and hold each other when the housing is folded over itself.

15. The method of claim 14, wherein the engaged geometric features irreversibly lock the housing in a closed position, thereby permanently identifying the line.

16. A kit for identifying a medical line comprising, a medical line coding device further comprising a housing and an illumination source; the housing having a first channel for the illumination source, and further comprising a second channel through which a medical line can pass, the kit further comprising suitable packaging material and instructions for using the device to identify a medical line.

17. The kit of claim 16, wherein the housing is capable of folding over itself.

18. The kit of claim 17, wherein the housing further comprises geometric features that engage and hold each other when the housing is folded over itself.

19. The kit of claim 18, wherein the engaged geometric features irreversibly lock the housing in a closed position.

20. The kit of claim 16, wherein the illumination source is chemiluminescent.

21. The kit of claim 16, wherein the illumination source provides light of a particular color.

22. The kit of claim 16, wherein the illumination source provides light of two or more particular colors.

23. The kit of claim 16, wherein the contents are sterile.

24. The kit of claim 23, wherein the packaging material provides a hermetic seal that indicates the sterility of the device contained therein.

25. A method of making a kit for identifying a medical line comprising, obtaining the medical line coding device of claim 1; obtaining suitable packaging material and instructions for using the device to identify a medical line, and incorporating the device into the packaging material.

26. The method of claim 25, wherein the kit includes a chemiluminescent illumination source.

27. The method of claim 26, wherein the illumination source provides light of a particular color.

28. The method of claim 26, wherein the illumination source provides light of two or more particular colors.

29. The method of claim 25, wherein the contents of the kit are sterilized.

30. A system for identifying a medical line comprising one or more medical line coding devices of claim 1, wherein the illumination source provides a color, which is interpretable as coding for a particular medication or fluid to be administered through the identified line.
